(19) United States
(12) Patent Application Publication
(10) Pub. No.: US 2025/0120641 A1
(43) Pub. Date: Apr. 17, 2025

(54) METHOD AND APPARATUS FOR SIMULTANEOUS COLLECTION OF SURFACE ELECTROMYOGRAPHY AND SKIN STRAIN FIELDS

(71) Applicant: Brigham Young University, Provo, UT (US)

(72) Inventors: Anton Bowden, Lindon, UT (US); David Fullwood, Provo, UT (US); Jordan Ryan, American Fork, UT (US); Alex Wonnacott, Provo, UT (US)

(21) Appl. No.: 18/916,479

(22) Filed: Oct. 15, 2024

Related U.S. Application Data
(60) Provisional application No. 63/544,297, filed on Oct. 16, 2023.

(51) Int. Cl. A61B 5/296; A61B 5/11; A61B 5/257; A61B 5/395

(52) U.S. Cl. CPC A61B 5/296; A61B 5/11; A61B 5/257; A61B 5/395; A61B 2560/0462; A61B 2562/0261; A61B 2562/043; A61B 2562/227

(57) ABSTRACT

A strain and stimulus sensor system includes stretchable sensors secured to a material configured to be worn by a user, a control system operably coupled to the sensors, and electrical connectors operably coupled to the control system. The sensors are configured to exhibit a change in electrical resistance as a function of mechanical deformation. Each of the electrical connectors is positioned on the sensor region to contact skin of the user and is configured to capture stimulus data. The control system is configured to send an electrical current through the sensors, measure an electrical resistance of the sensors responsive to the electrical current, and measure an electrical activity at the skin from the electrical connectors contacting the skin.

### CROSS-REFERENCE TO RELATED APPLICATIONS

This application claims priority to U.S. Provisional Patent Application No. 63/544,297 filed on Oct. 16, 2023, the disclosure of which is incorporated herein, in its entirety, by this reference.

### BACKGROUND

Surface electromyography (sEMG) can be used to distinguish between muscle behavior in subject, and sensors have been used to collect motion (kinematic) data from subject. Despite the obvious advantages of including both kinematics and sEMG as tools for clinical diagnosis, including either metric in clinical practice is rare, due to the high costs of clinically relevant systems as well as the specialized expertise required to interpret the results.

### SUMMARY

Embodiments disclosed herein include a strain and stimulus sensor system. The system may include a first material including a sensor region and configured to be worn by a user, a plurality of stretchable sensors secured to the first material in the sensor region, a control system including a circuit board and one or more wires secured or securable to the plurality of stretchable sensors and operably connected to the circuit board, and a plurality of electrical connectors secured to the plurality of stretchable sensors and at least one wire of the one or more wires. The plurality of sensors are configured to exhibit a change in electrical resistance as a function of mechanical deformation. Each electrical connector of the plurality of electrical connectors is positioned on the sensor region to contact skin of the user and configured to capture stimulus data. The control system is configured to send an electrical current through the plurality of sensors, measure an electrical resistance of the plurality of stretchable sensors responsive to the electrical current, and measure an electrical activity at the skin from one or more electrical connectors of the plurality of electrical connectors contacting the skin.

In an embodiment, a method of collecting strain and stimulus data from a selected region of a body of a user is disclosed. The method includes securing a first material to the body of a user such that a plurality of electrical connectors secured to the first material contact skin in the selected region of the body. The first material includes a sensor region having a plurality of stretchable sensors secured thereto with the plurality of electrical connectors. The method also includes sending an electrical current from a circuit board, through one or more wires operably connected to the circuit board and the plurality of electrical connectors, and through the plurality of stretchable sensors. The method also includes measuring an electrical resistance of the plurality of stretchable sensors responsive to the electrical current. The plurality of sensors exhibit a change in electrical resistance as a function of mechanical deformation. The method also includes measuring an electrical activity at the skin from one or more electrical connectors of the plurality of electrical connectors contacting the skin. The plurality of electrical connectors capture stimulus data responsive to the electrical current.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 shows a front view of a sensor arrangement, according to an embodiment.

FIG. 2 shows a top view of a first portion of a wearable garment including sensors, according to an embodiment.

FIG. 3 shows a top view of a second portion of the wearable garment including a control system and one or more wires, according to an embodiment.

FIG. 4 shows a top view of the first portion of the wearable garment of FIG. 2 secured to the second portion of the wearable garment of FIG. 3.

FIG. 5 is a schematic of a controller, according to an embodiment.

FIG. 6 is a flow diagram of a method of collecting strain and stimulus data from a selected region of a body of a user, according to an embodiment.

### DETAILED DESCRIPTION

Embodiments disclosed herein include systems and methods having a wearable device that collects substantially simultaneously both skin strain data (e.g., motion data) and surface electromyography (sEMG) data (e.g., stimulus data) for any given or selected region of the body. In contrast to conventional systems that requires separate devices with separate and burdensome data processing, systems and methods disclosed herein allow for simultaneous collection of these data that provides simultaneous information on the movements (e.g., kinematics) and muscle activation of the user wearing the device. In many embodiments, the systems and methods disclosed herein are configured to collect field data for both the skin strain and the sEMG, rather than single location measurements that are typically collected. Thus, in many embodiments, kinematics and muscle activation for an entire region of the body can be collected simultaneously, which is challenging or impossible using conventional systems.

The data collected from the systems and methods disclosed herein including a wearable device may be useful in classifying and phenotyping the movement profile of the user wearing the wearable device, which may be predictive of one or more of: a) the health status of the wearer; b) improvements and changes over time in the health status of the wearer; c) biomechanical features that may predict the most effective clinical intervention for the wearer based on an underlying medical condition (e.g., chronic low back pain, knee pain, shoulder pain, etc.); d) the technique used by the wearer in sporting events; and/or e) improvements and changes over time in the technique used in sporting events by the wearer.

In at least one embodiment, the collected data is collected from the sensors using a control system including, for example, a small printed circuit board (PCB). The data may be stored locally on the PCB or passed via Bluetooth to a nearby smartphone, and then the data may be passed into a previously trained machine learning algorithm which performs data analysis and returns user-interpretable information and recommendations back to the smartphone for display and/or recording.

Systems and methods disclosed herein may provide for simultaneously reading of sEMG and skin strain data. This skin strain data can be used as a surrogate for determining joint kinematics data. The wearable device may be integrated with a wearable material, allowing for placement of the device to be repeatable and consistent due to established markers for correct orientation. This repeatability of placement of the device of the systems and methods disclosed herein allows for finding the distinctions between sEMG signals and deep muscle readings. In many embodiments, the systems and method disclosed herein use only one PCB for collection of both the sEMG data and the skin strain data.

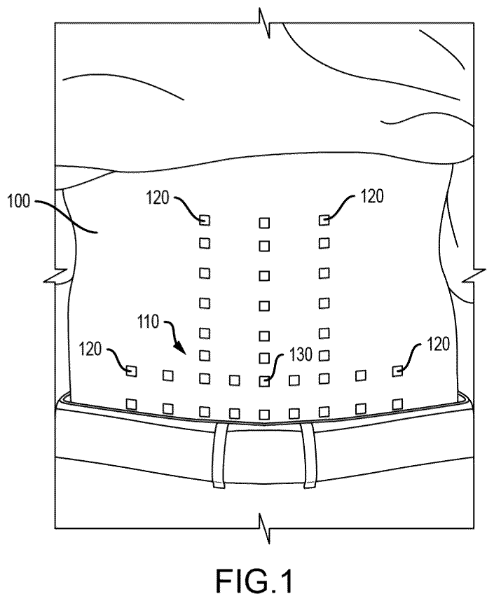

FIG. 1 shows a front view of a sensor orientation 110 on a back of a user 100, according to an embodiment. In one or more embodiments, motion capture may be used to identify a very dense marker set of a sensor orientation 110 configured to allow the wearable device to collect high-density skin strain field during motion. In FIG. 1, the sensor orientation is positioned on a lumbar region. The systems and methods disclosed herein, however, may utilize motion capture to identify marker sets of sensor orientations of other regions of the body. In some embodiments, the sensor orientation may identify sensor points 120 and a bony prominence 130.

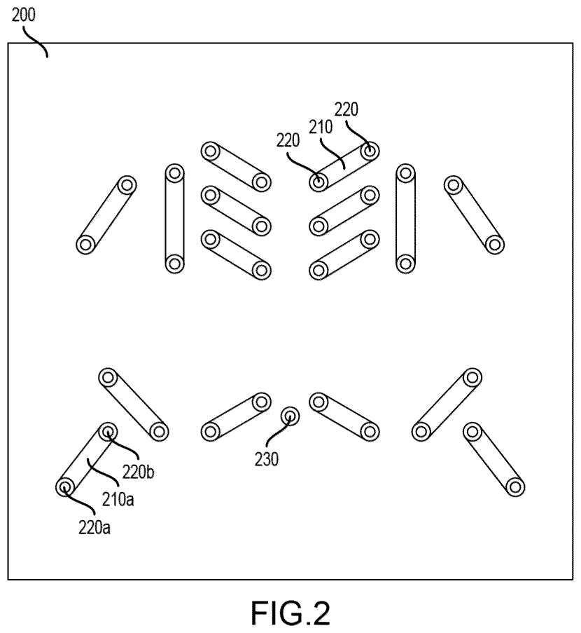

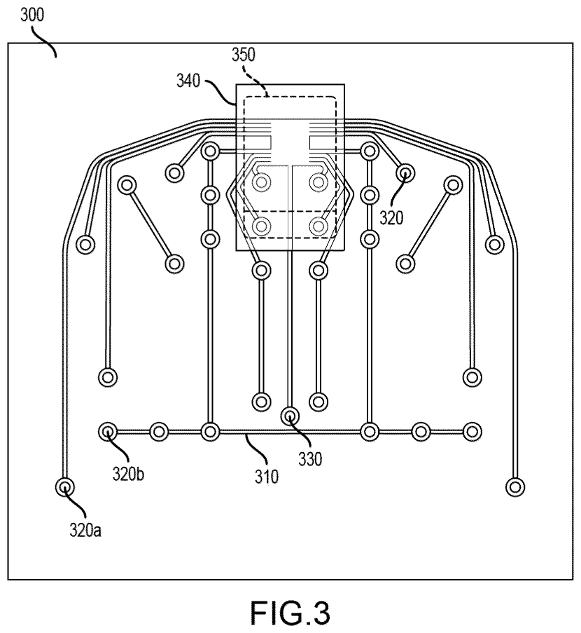

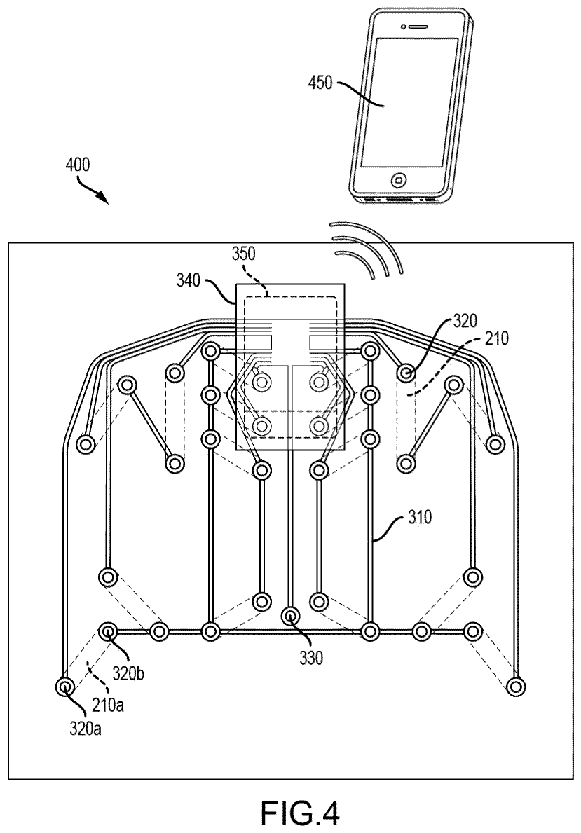

FIGS. 2-4 show aspects of a strain and stimulus sensor system, according to an embodiment. FIG. 2, for example, shows a top view of a first portion 200 of a wearable material of the strain and stimulus sensor system including a plurality of sensors 210 and a first connector portion 220 of electrical connectors, according to an embodiment. FIG. 3 shows a top view of a second portion 300 of the wearable material including a control system, one or more wires 310, and a second connector portion 320 of electrical connectors, according to an embodiment. FIG. 4 shows a top view of the first portion 200 of the wearable material of FIG. 2 secured to the second portion 300 of the wearable material of FIG. 3. At least a portion of the wearable material may include a stretchable fabric base material, such as kinesiology tape.

In some embodiments, the first portion 200 of the wearable material includes a stretchable fabric base material configured to adhere and/or fit tightly against a region of the body such as spandex, elastane, stretch cotton, nylon, mesh, Lycra, and/or combinations thereof. The material of the second portion 300 of the wearable material may be the same as or different than the first portion 200 of the wearable material. As shall be discussed in greater detail below, the second portion 300 may be detachably secured to the first portion 200 of the wearable material. Accordingly, the first portion 200 of the wearable material may include the stretchable fabric base material, but the second portion 300 may include the same or different materials that are not necessarily a stretchable fabric. In many embodiments, the material of the second portion 300 of the wearable material may include type of fabric, paper, or deformable sheet.

Turning to FIG. 2, the plurality of sensors 210 may be stretchable and secured to a sensor region of the first portion 200 of the wearable material. In some embodiments, the plurality of sensors 210 may include nanocomposite stretch sensors mounted on the first portion 200 to electrically insulate the plurality of sensors 210 from the skin on the back of the user 100. For example, the plurality of sensors 210 may be positioned on a surface of the first portion 200 that is opposite to the skin of the user when worn by the user. In some embodiments, the plurality of sensors 210 may be embedded within the first portion 200 of the wearable material. In these and other embodiments, at least some of the first portion 200 of the wearable material is positioned between the plurality of sensors 210 and the skin of the user 100 when the first portion 200 is worn by the user 100.

The plurality of sensors 210 are configured to exhibit a change in electrical resistance as a function of mechanical deformation. For example, as a sensor 210 is stretching, the resistance of the sensor changes. In some embodiments, the plurality of sensors 210 includes multilayered sensors. In some embodiments, each of the plurality of sensors 210 include a plurality of strain gauges. More particularly, the plurality of sensors 210 may include strain gauges screen printed onto the first portion 220 of the wearable material. Screen printing ink, for example, may be blended with metal nano-particles to form a composite material that is screen printed on the first portion of the wearable material in a predetermined pattern to be the plurality of sensors 220. The metal nano-particles may include nickel coated carbon fibers and nickel nanostrands, and the screen printing ink may include a silicone ink base. In some embodiments, the composite material of screen printing ink and metal nano-particles includes greater than about 12% saturation of the metal nano-particles. In some embodiments, the composite material includes at least about 5% saturation of the metal nano-particles, at least about 10% saturation of the metal nano-particles, at least about 15% saturation of the metal nano-particles, at least about 20% saturation of the metal nano-particles, at least about 25% saturation of the metal nano-particles, at least about 30% saturation of the metal nano-particles, about 5% to about 25% saturation of the metal nano-particles, about 5% to about 10% saturation of the metal nano-particles, about 10% to about 15% saturation of the metal nano-particles, about 15% to about 20% saturation of the metal nano-particles, about 20% to about 25% saturation of the metal nano-particles, or about 20% to about 25% saturation of the metal nano-particles.

The composite material also may include about 5 wt % to about 8 wt % of catalyst. In some embodiments, the composite material may include about 2 wt % to about 16 wt % of catalyst, about 2 wt % to about 9 wt % of catalyst, about 9 wt % to about 16 wt % of catalyst, about 4 wt % to about 9 wt % of catalyst, about 3 wt % to about 10 wt % of catalyst, about 5 wt % to about 7 wt %, about 6 wt % to about 8 wt % of catalyst, about 4 wt % of catalyst, about 5 wt % of catalyst, about 6 wt % of catalyst, about 7 wt % of catalyst, about 8 wt % of catalyst, about 9 wt % of catalyst, or about 10 wt % of catalyst.

When printed on the first portion 220 of the wearable material, the plurality of sensors 210 may have a thickness of less than about 1.0 mm, such as about 0.75 mm, about 0.6 mm, about 0.5 mm, about 0.4 mm, about 0.3 mm, less than about 0.3 mm, about 0.1 mm to about 1 mm, about 0.1 mm to about 0.5 mm, about 0.5 mm to about 1.0 mm, about 0.1 mm to about 0.3 mm, about 0.3 mm to about 0.5 mm, about 0.5 mm to about 0.7 mm, or about 0.7 mm to about 0.9 mm. In some embodiments, the plurality of sensors 210 may include strain gauges secured to the first portion 200 of the wearable material with adhesive, stitching, ultrasonic welding, or combinations thereof. Other sensors and positioning of sensors are disclosed in U.S. Pat. No. 9,857,246, the disclosure of which is incorporated herein by reference in its entirety.

In many embodiments, the plurality of sensors 210 are spaced from one another and oriented on the first portion 200 of the wearable material based on the sensor orientation 110 and sensor points 120 identified, for example, through motion capture. The number of the plurality of sensors 210 also may be at least partially based on the sensor orientation 110 and sensor points 120 identified, for example, through motion capture. The number of sensors in the plurality of sensors 210, the positioning of the plurality of sensors 210, and/or the orientation of the plurality of sensors 210 in the first portion 200 of the wearable material may be selected to collect appropriate kinematic information. For example, as noted above, in FIG. 1 the sensor orientation is positioned on a lumbar region. In an example of the system, it was found that using sixteen sensors on the skin of the lumbar spine allows for collection of the underlying kinematics of the L1, L2, L3, L4, and LS vertebrae.

The first portion 200 of the wearable material also includes a plurality of first connector portions 220 or parts of a plurality of electronical connectors positioned on the first portion 200 of the wearable material to contact skin of the user 100 and capture stimulus data. Each of the plurality of sensors 210 may include at least one first connector portion 220 passing or extending therethrough. Accordingly, each first connector portion 220 may pass through at least one sensor 220 and the first portion 200 of the wearable material such that the first connector portion contacts the skin of the user 100 when the wearable material is worn by the user 100. In some embodiments, each sensor of the plurality of sensors 210 may include a single first connector portion 220 or multiple connector portions 220 extending therethrough.

The first portion 200 of the wearable material also may include an additional connector portion 230 or part of an electrical connector. In many embodiments, the additional connector portion 230 does not pass or extend through any sensor of the plurality of sensors 210. Accordingly, the additional connector portion 230 may extend or pass through only the first portion 200 of the wearable material. The additional connector portion 230 may act in the system as a ground reference electrical lead. For example, the additional first connector portion 230 (e.g., additional electrical lead) may be used to determine a localized ground signal from the body that will compare against stimuli signals from measured areas, such as where the first connector portions 220 contact the skin. The additional first connector portion 230 is positioned on the bony prominence because the bony prominence causes a region where less electrical activity occurs and can therefore be used as a reference point for signal processing. The additional connector portion 230 is positioned on the first portion 200 of the wearable material to contact the skin of the user at a bony prominence of the user 100. For example, in the lumbar spine, the additional connector portion 230 may be positioned to be located on the skin of the user 100 directly above the spinous process of the LS vertebra.

The plurality of first connector portions 220 and the additional connector portion 230 may include a metallic material or other electrically conductive material. In some embodiments, the plurality of first connector portions 220 and the additional connector portion 230 are configured to detachably secure to a plurality of second connector portions 320 (described in greater detail below) and an additional second connector portion 330. For example, the plurality of first connector portions 220 and the plurality of second connector portions 320 may include conductive snap connectors configured to selectively snap or attach together to form a sEMG lead (e.g., electrical connector or electrode). Similarly, the additional first connector portion 230 and the additional second connector portion 330 may include a conductive snap connector configured to selectively snap or attach together to form a sEMG lead (e.g., electrical connector or electrode).

Turning now specifically to FIG. 3, which shows the second portion 300 of wearable material. As noted above, the second portion 300 of wearable material may include the same or different material than the first portion 200 of wearable material. The second portion 300 of wearable material may be distinct and separate from the first portion 200 of wearable material. The second portion 300 of wearable material may include the control system, the one or more wires 310, the second connector portions 320 of electrical connectors, and/or the additional second connector portion 330 of the electrical connector. In these and other embodiments, the second portion 300 of wearable material may be configured to detachably or fixedly secure or attach to the first portion 200 of wearable material.

In many embodiments, the plurality of second connector portions 320 are positioned to align with and connect to the plurality of first connector portions 220 on the first portion 200 of wearable material, as shown in FIG. 4. For example, second connector portions 320a and 320b are positioned on the second portion 300 of wearable material to connect to the first connector portions 220a and 220b on the first portion 200 of wearable material. Similarly, the additional second connector portion 330 is positioned on the second portion 200 of wearable material to align with and connect to the additional first connector portion 230 on the first portion 200 of wearable material. With the second connector portions 320a and 320b connected to the first connector portions 220a and 220b on the first portion 200 of wearable material, electrical leads for capturing sEMG data are formed. In the lumbar spine example, sixteen electrical leads are formed from the first connector portions 220 and the second connector portions 320, and an additional ground reference electrical lead from the additional first connector portion 230 and the additional second connector portion 330.

The second portion also includes a control system including a circuit board 350 and one or more wires 310 secured to the circuit board 350, the plurality of second connector portions 320, and the additional second connector portion 330. With the one or more wires 310 secured to the plurality of second connector portions 320 and the additional second connector portion 330, the one or more wires 310 are configured to send information to the circuit board 350 from the plurality of sensors 210, the plurality of first connector portions 220, and the additional first connector portion 230 when the plurality of first connector portions 220 and the additional first connector portion 230 are secured to the plurality of second connector portions 320, and the additional second connector portion 330 to form the electrical connectors. The control system is configured to send an electrical current through the one or more wires 310 to the plurality of sensors 210, measure an electrical resistance of the plurality of sensors 210 responsive to the electrical current, and measure an electrical activity at the skin from the plurality of first connector portions 220 and the additional first connector portion 230 contacting the skin.

While the first portion 200 may be separate and distinct from the second portion 300 of wearable material, in some embodiments, the first portion 200 of wearable material includes the control system, the one or more wires 310, the second connector portions 320 of electrical connectors, and the additional second connector portion 330 of the electrical connector, and the second portion 300 of wearable material is absent. In these and other embodiments, the first connector portion 220 and the second connector portion 230 may include a single, continuous electrical connector, and the additional first connector portion 230 and the additional second connector portion 330 may form a single continuous electrical connector.

The one or more wires 310 may include a conductive wire 310 connected to the circuit board 350, the second connector portions 320, and the additional second connector portion 330. The circuit board 350 may include a communication module configured to communicate with one or more controllers or a memory/storage device. For example, readings and/or data from one or more of the plurality of sensors 110 may be transmitted via BLUETOOTH, other wireless communication, or wired communication to a nearby electronic device 450, such as a smartphone, tablet computer, laptop computer, or desktop computer. In some embodiments, the circuit board 350 is secured to at least one of the first portion 200 or the second portion 300 of wearable material. In some embodiments, the second portion 300 includes a pocket 340 configured to hold and secure the circuit board 350 to the second portion 300 of wearable material. In some embodiments, the circuit board 350 is detachably connected to the conductive wire 310, the electrical connectors, and the plurality of sensors 310. In some embodiments, the circuit board 350 is programmed to collect the data from the electrical connectors and the plurality of sensors 210, is connected through a micro-HDMI port, and transfers the data via Bluetooth and Wi-Fi to an online database for storage.

In some embodiments, the circuit board 350 is configured to communicate with the electronic device 450. The electronic device 450, accordingly, may be configured to communicate with the circuit board 450 and receive from the circuit board the stimulus data at the bony prominence from the additional first connection portion 230, the electrical resistance of the plurality of stretchable sensors 210 responsive to the electrical current, and the electrical activity at the skin from one or more of the plurality of first connection portions 220 contacting the skin. The electronic device 450 also may be configured to determine a strain and a stimulus in the region of the body of the user based on the electrical resistance of the plurality of stretchable sensors 210 responsive to the electrical current, the electrical activity at the skin from one or more of the plurality of first connection portions 220 contacting the skin, and the electrical activity at skin at the bony prominence from the additional first connection portion 230.

The circuit board 350 also may be configured to send, in time cycles and at selected currents, an electrical current through the one or more wires 310 to the plurality of sensors 210, measure an electrical resistance of the plurality of sensors 210 responsive to the electrical current, and measure an electrical activity at the skin from the plurality of first connector portions 220 and the additional first connector portion 230 contacting the skin. In some embodiments, the circuit board 350 and the control system are configured to alternate rapidly between an active mode and a passive mode. This alternating between the active mode and the passive mode may occur, for example, thousands of times per second.

Time cycling of the data collection from each of the electrical connectors (including the first connector portions 220 and the additional first connector portion 230 acting as electrical leads), first in an active mode which sequentially sends a small electrical current through each of the plurality of sensors 210 in the array and measures the instantaneous resistance of each sensor with respect to the common electrical ground of the PCB 350. The common electrical ground is the ground line to which the plurality of sensors 210 for mobility are wired (i.e., the plurality of sensors 210 are all measured with respect to a common reference voltage). Each sensor of the plurality of sensors 210 may include two electrodes or first connector portions 220 secure thereto: one is connected back to the PCB pin for measurement, and the other is connected to the ground wire for the entire electrical system (i.e., the common ground).

Secondly, in a passive mode, the electrical activity (e.g., the sEMG data) on the surface of the skin at the location of each of the first connector portion 220 and the additional first connector portion 230 is sequentially measured (the first connector portions 220 and the additional first connector portion 230 forming electrical leads with the second connector portions and the additional second connector portion 330, respectively). This sEMG data may indicate the electrical signal coming from the muscles underlying the first connector portion 220. For example, the resistances of the plurality of sensors 210 can be measure at a frequency of 50 Hz for several milliseconds each, leaving a time gap in between sampling from each sensor of the plurality of sensors 210, and the sEMG data at each of the first connector portions 220 and the additional first connector portion 230 can be measured at a frequency of 300 Hz for several milliseconds each, again leaving a time gap between sampling from each of the first connector portions 220 and the additional first connector portion 230. The electrical leads formed by the connector portions 220, 230, 320, 330 allow the circuit board 350 or control system to read the sEMG signals between pulses sent by the circuit board 350. Frequencies that are not within the sEMG dataset range may be filtered out from the sEMG data.

Accordingly, the control system (e.g., the circuit board 350) may be configured to send a first electrical current at a first selected frequency and send a second electrical current at a second selected frequency different from the first selected frequency through the plurality of sensors 210. The control system then may be further configured to measure a first electrical resistance of the plurality of sensors 210 responsive to the first electrical current, measure a second electrical resistance of the plurality of sensors 210 responsive to the second electrical current, measure a first electrical activity at the skin from one or more of the plurality of first connector portions 220 contacting the skin between sending the first electrical current and the second electrical current, and measure a second electrical activity at the skin from one or more of the first connector portions 220 contacting the skin after sending the second electrical current.

The strain and stimulus sensor system including the first portion 200 and the second portion 300 of wearable material provides an inexpensive wearable system that measures biomechanical features of patient kinematics and, in some embodiments, uses a machine-learning approach to identify likely effective treatment phenotypes. By utilizing the plurality of sensors 210 positioned in an array over the select region of the body of the user 100, the strain and stimulus sensor system can distinguish segmental level motion characteristics. Moreover, the strain and stimulus sensor system can utilize the first connector portions 220 secured to the plurality of sensors 210 and contacting the skin of the user 100 to enable simultaneous collection of muscle activation signals (EMG data) from the paraspinal musculature. The combination of the plurality of sensors 210 and the first connector portions secured to the plurality of sensors 210 and contacting the skin of the user 100 allows the system to be multi-modal, providing a more comprehensive suite of musculoskeletal information that could enhance the specificity of the phenotyping information provided by the technology. Muscle activation information is especially interesting in the context of altered muscle recruitment patterns and fear avoidance behaviors, both of which are common to chronic lower back pain (cLBP) individuals. Muscle activation signals are especially relevant to surgical and physical therapy treatment paradigms.

While a lumbar example has been provided above, the strain and stimulus sensor system disclosed herein may be applied to numerous fields of medical evaluation and function tracking. In the example application in the field of spinal biomechanics and cLBP, individuals with cLBP are known to exhibit their own “spinal movement signature” and possibly even specific altered gait patterns. These changes from “normal” are most likely a function of altered motor control, central sensitization and kinesiophobia and are noticeable in both their kinematic motion and their muscle activation signals (as measured using sEMG). In particular, the activity of two back muscle groups, the erector spinae and multifidus, have been found to exhibit bioelectrical changes in patients with cLBP compared to people without.

The combination of simultaneously collecting motion (kinematic) data from the plurality of sensors 210 while also collecting sEMG from the first connector portions 220 contacting the skin and secured to the plurality of sensors 210 is useful. Motion data are helpful for providing information of joint ranges-of-motion in space and time, while sEMG signals specify the timing of muscle activation and provide an estimate on muscle force production. By combining these measures together in the systems disclosed herein, a more complete picture of spinal movements and their limitations is provided in contrast to using either of sEMG signals or motion data on their own. The systems disclosed herein provide an inexpensive, wearable technology that provides the motion data and the sEMG data. Integration and time-synchronization of sEMG and kinematics data using conventional systems is a complex and expensive process. In many embodiments, firmware-based rapid switching of sampling between sEMG voltages and nanocomposite sensor resistances is provided, both muscle activation data and kinematics data are automatically synchronized, and data acquisition rates exceeding 100 Hz is supported. Analysis of the sEMG data from the systems disclosed herein may be integrated into a cloud-based machine learning network that automatically interprets the data and returns phenotyping information relevant for clinical decision-making.

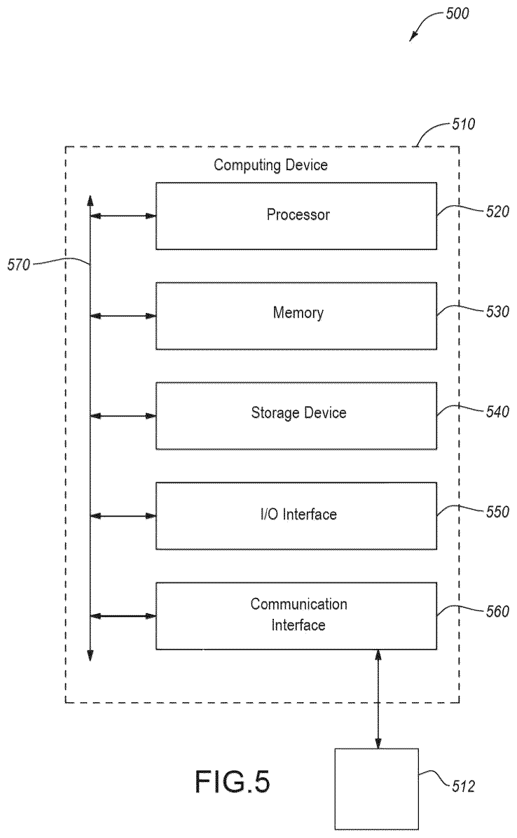

FIG. 5 is a schematic of a controller 500 for executing any of the example methods and systems disclosed herein, according to an embodiment. The controller 500 includes at least one computing device 510. The at least one computing device 510 is an exemplary computing device that may be configured to perform one or more of the acts described above. For example, the at least one computing device 510 may include the PCB and/or the electronic device 450. The at least one computing device 510 can include one or more servers, one or more computers (e.g., desk-top computer, lap-top computer), or one or more mobile computing devices (e.g., smartphone, tablet, etc.). The computing device 510 can comprise at least one processor 520, memory 530, a storage device 540, an input/output (“I/O”) device/interface 550, and a communication interface 560. While an example computing device 510 is shown in FIG. 5, the components illustrated in FIG. 5 are not intended to be limiting of the controller 500 or computing device 510. Additional or alternative components may be used in some examples. Further, in some examples, the controller 500 or the computing device 510 can include fewer components than those shown in FIG. 5. For example, the controller 500 may not include the one or more additional computing devices 512. In some examples, the at least one computing device 510 may include a plurality of computing devices, such as a server farm, computational network, or a cluster of computing devices. Components of computing device 510 shown in FIG. 5 are described in additional detail below.

In some examples, the processor(s) 520 includes hardware for executing instructions (e.g., instructions for carrying out one or more portions of any of the methods and systems disclosed herein), such as those making up a computer program. For example, to execute instructions, the processor(s) 520 may retrieve (or fetch) the instructions from an internal register, an internal cache, the memory 530, or a storage device 540 and decode and execute them. As an example, the processor(s) 520 may include one or more instruction caches, one or more data caches, and one or more translation lookaside buffers (TLBs). Instructions in the instruction caches may be copies of instructions in memory 530 or storage device 540. In some examples, the processor 520 may be configured (e.g., include programming stored thereon or executed thereby) to carry out one or more portions of any of the example methods disclosed herein.

In some examples, the processor 520 is configured to perform any of the acts disclosed herein or cause one or more portions of the computing device 510 or controller 500 to perform at least one of the acts disclosed herein. Such configuration can include one or more operational programs (e.g., computer program products) that are executable by the at least one processor 520.

The at least one computing device 510 (e.g., a server) may include at least one memory storage medium (e.g., memory 530 and/or storage device 540). The computing device 510 may include memory 530, which is operably coupled to the processor(s) 520. The memory 530 may be used for storing data, metadata, and programs for execution by the processor(s) 520. The memory 530 may include one or more of volatile and non-volatile memories, such as Random Access Memory (RAM), Read Only Memory (ROM), a solid state disk (SSD), Flash, Phase Change Memory (PCM), or other types of data storage. The memory 530 may be internal or distributed memory.

The computing device 510 may include the storage device 540 having storage for storing data or instructions. The storage device 540 may be operably coupled to the at least one processor 520. In some examples, the storage device 540 can comprise a non-transitory memory storage medium, such as any of those described above. The storage device 540 (e.g., non-transitory storage medium) may include a hard disk drive (HDD), a floppy disk drive, flash memory, an optical disc, a magneto-optical disc, magnetic tape, or a Universal Serial Bus (USB) drive or a combination of two or more of these. Storage device 540 may include removable or non-removable (or fixed) media. Storage device 540 may be internal or external to the computing device 510. In some examples, storage device 540 may include non-volatile, solid-state memory. In some examples, storage device 540 may include read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. In some examples, one or more portions of the memory 530 and/or storage device 540 (e.g., memory storage medium(s)) may store one or more databases thereon.

In some examples, sensed strain from the plurality of sensors may be stored in a memory storage medium such as one or more of the at least one processor 520 (e.g., internal cache of the processor), memory 530, or the storage device 540. In some examples, the at least one processor 520 may be configured to access (e.g., via bus 570) the memory storage medium(s) such as one or more of the memory 530 or the storage device 540. For example, the at least one processor 520 may receive and store the data (e.g., look-up tables) as a plurality of data points in the memory storage medium(s). The at least one processor 520 may execute programming stored therein adapted to access the data in the memory storage medium(s) to automatically determine a likely source of a sensed strain by the plurality of sensors.

The computing device 510 also includes one or more I/O devices/interfaces 550, which are provided to allow a user to provide input to, receive output from, and otherwise transfer data to and from the computing device 510. These I/O devices/interfaces 550 may include a mouse, keypad or a keyboard, a touch screen, camera, optical scanner, network interface, web-based access, modem, a port, other known I/O devices or a combination of such I/O devices/interfaces 550. The touch screen may be activated with a stylus or a finger.

The I/O devices/interfaces 550 may include one or more devices for presenting output to a user, including, but not limited to, a graphics engine, a display (e.g., a display screen or monitor), one or more output drivers (e.g., display drivers), one or more audio speakers, and one or more audio drivers. In certain examples, I/O devices/interfaces 550 are configured to provide graphical data to a display for presentation to a user. The graphical data may be representative of one or more graphical user interfaces and/or any other graphical content as may serve a particular implementation.

The computing device 510 can further include a communication interface 560. The communication interface 560 can include hardware, software, or both. The communication interface 560 can provide one or more interfaces for communication (such as, for example, packet-based communication) between the computing device 510 and one or more additional computing devices 512, 514 or one or more networks. For example, the communication interface 560 may include a network interface controller (NIC) or network adapter for communicating with an Ethernet or other wire-based network or a wireless NIC (WNIC) or wireless adapter for communicating with a wireless network, such as a WI-FI. The additional computing device 512 may include the communication module 140, and the communication module 140 may include any aspect or component of the controller 500.

Any suitable network and any suitable communication interface 560 may be used. For example, the computing device 510 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, one or more portions of controller 500 or the computing device 510 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination thereof. The computing device 510 may include any suitable communication interface 560 for any of these networks, where appropriate.

The computing device 510 may include a bus 570. The bus 570 can include hardware, software, or both that couples components of computing device 510 to each other. For example, bus 570 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination thereof.

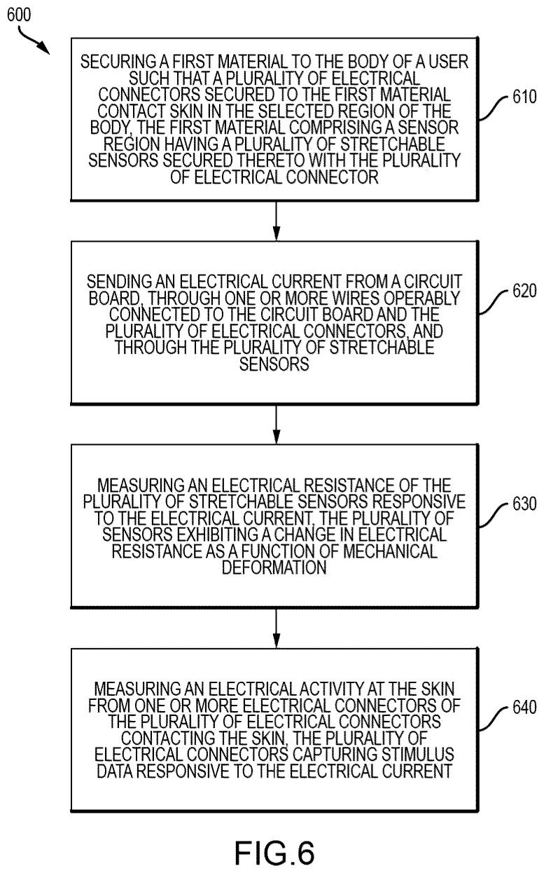

FIG. 6 is a flow diagram of a method 600 of collecting strain and stimulus data from a selected region of a body of a user, according to an embodiment. The method 600 may utilize any of the systems disclosed herein. In an embodiment, the method 600 includes securing 610 a first material to the body of a user such that a plurality of electrical connectors secured to the first material contact skin in the selected region of the body. The first material includes a sensor region having a plurality of stretchable sensors secured thereto with the plurality of electrical connectors. The method 600 includes sending 620 an electrical current from a circuit board, through one or more wires operably connected to the circuit board and the plurality of electrical connectors, and through the plurality of stretchable sensors. The method 600 includes measuring 630 an electrical resistance of the plurality of stretchable sensors responsive to the electrical current. The plurality of sensors exhibit a change in electrical resistance as a function of mechanical deformation. The method 600 also includes measuring 640 an electrical activity at the skin from one or more electrical connectors of the plurality of electrical connectors contacting the skin. The plurality of electrical connectors capture stimulus data responsive to the electrical current.

In some embodiments of the method 600, the first material comprises an adhesive and stretchable fabric material. In some embodiments, the method 600 also may include motion capturing the selected region of the body of the user and identifying a plurality of locations for the plurality of sensors based on the motion capturing. The plurality of sensors may be spaced from one another in the sensor region and the plurality of electrical connectors are spaced from one another in the sensor region.

In some embodiments, the method 600 further comprises positioning an additional electrical connector to contact a bony prominence of the user. The additional electrical connector is secured to the first material and at least one wire of the one or more wires. The method 600 also may include measuring an electrical activity at the bony prominence from the additional electrical connector contacting the bony prominence.

In some embodiments, the method 600 includes transmitting, from the circuit board, the stimulus data at the bony prominence, the electrical resistance of the plurality of stretchable sensors responsive to the electrical current, and the electrical activity at the skin from one or more of the plurality of electrical connectors contacting the skin to an electronic device.

The method 600 also may include determining, with the electronic device, a strain and a stimulus on the user based on the electrical resistance, the electrical activity at the skin from the one or more of the plurality of electrical connectors, and the electrical activity at the skin from the additional electrical connector. In these and other embodiments of the method 600 sending an electrical current from a circuit board, through one or more wires operably connected to the circuit board and the plurality of electrical connectors, and through the plurality of stretchable sensors may include sending a first electrical current at a first selected frequency through the plurality of stretchable sensors and sending a second electrical current at a second selected frequency different from the first selected frequency through the plurality of stretchable sensors. In these and other embodiments of the method 600, measuring an electrical resistance of the plurality of stretchable sensors responsive to the electrical current may include measuring a first electrical resistance of the plurality of stretchable sensors responsive to the first electrical current and measuring a second electrical resistance of the plurality of stretchable sensors responsive to the second electrical current. In many embodiments of the method 600, measuring an electrical activity at the skin from one or more electrical connectors of the plurality of electrical connectors contacting the skin may include measuring a first electrical activity at the skin from one or more of the plurality of electrical connectors contacting the skin between sending the first electrical current and the second electrical current and measuring a second electrical activity at the skin from one or more of the plurality of electrical connectors contacting the skin after sending the second electrical current.

In some embodiments, the method 600 also includes, for each electrical connector of the plurality of electrical connectors, detachably securing a first connector portion to a second connector portion to form an electrical connection between the first connector portion and the second connector portion. The first connector portion may secure a stretchable sensor of the plurality of stretchable sensors to the first material with at least some of the first connector portion contacting the skin of the user and the second connector portion is secured to at least one wire of the one or more wires. The method 600 also may include detachably securing an additional first connector portion of the additional electrical connector to an additional second connector portion of the additional electrical connector to form an electrical connection between the additional first connector portion and the additional second connector portion. The first connector portion may be secured to the first material and contacting the bony prominence of the user, and the additional second connector portion may be secured to at least one wire of the one or more wires. In these and other embodiments, the second connector portion of the plurality of electrical connectors and the additional second connector portion of the additional electrical connector may be secured to a second material such that detachably securing the first connector portion to the second connector portion and detachably securing the additional first connector portion of the additional electrical connector to the additional second connector portion detachably secures the second material to the first material. The one or more wires may be secured to the second material.

Acts of the method 600 are for illustrative purposes. For example, the acts of the method 600 may be performed in different orders, split into multiple acts, modified, supplemented, or combined.

As used herein, the term “about” or “substantially” refers to an allowable variance of the term modified by “about” by ±10% or ±5%. Further, the terms “less than,” “or less,” “greater than”, “more than,” or “or more” include as an endpoint, the value that is modified by the terms “less than,” “or less,” “greater than,” “more than,” or “or more.”

Features from any of the disclosed embodiments may be used in combination with one another, without limitation. In addition, other features and advantages of the present disclosure will become apparent to those of ordinary skill in the art through consideration of the following detailed description and the accompanying drawings.

While various aspects and embodiments have been disclosed herein, other aspects and embodiments are contemplated. The various aspects and embodiment disclosed herein are for purposes of illustration and are not intended to be limiting.

What is claimed is:

1. A strain and stimulus sensor system, comprising:
a first material including a sensor region and configured to be worn by a user;
a plurality of sensors that are stretchable and secured to the first material in the sensor region, the plurality of sensors configured to exhibit a change in electrical resistance as a function of mechanical deformation;
a control system including a circuit board and one or more wires secured or securable to the plurality of sensors and operably connected to the circuit board;
a plurality of electrical connectors secured to the plurality of sensors and at least one wire of the one or more wires, each electrical connector of the plurality of electrical connectors positioned on the sensor region to contact skin of the user and configured to capture stimulus data; and
wherein the control system is configured to send an electrical current through the plurality of sensors, measure an electrical resistance of the plurality of sensors responsive to the electrical current, and measure an electrical activity at the skin from one or more electrical connectors of the plurality of electrical connectors contacting the skin.

2. The strain and stimulus sensor system of claim 1, further comprising an additional electrical connector secured to the sensor region and at least one wire of the one or more wires, the additional electrical connector positioned to contact a bony prominence of the user and configured to capture stimulus data at the bony prominence.

3. The strain and stimulus sensor system of claim 2, further comprising an electronic device configured to:
communicate with the circuit board and receive from the circuit board the stimulus data at the bony prominence, the electrical resistance of the plurality of sensors responsive to the electrical current, and the electrical activity at the skin from one or more of the plurality of electrical connectors contacting the skin; and
determine a strain and a stimulus on the user based on the electrical resistance, the electrical activity at the skin from the one or more of the plurality of electrical connectors, and the electrical activity at the skin from the additional electrical connector.

4. The strain and stimulus sensor system of claim 3, wherein the control system is configured to send a first electrical current at a first selected frequency and send a second electrical current at a second selected frequency different from the first selected frequency through the plurality of sensors.

5. The strain and stimulus sensor system of claim 4, wherein the control system is configured to:
measure a first electrical resistance of the plurality of sensors responsive to the first electrical current;
measure a second electrical resistance of the plurality of sensors responsive to the second electrical current;
measure a first electrical activity at the skin from one or more of the plurality of electrical connectors contacting the skin between sending the first electrical current and the second electrical current; and
measure a second electrical activity at the skin from one or more of the plurality of electrical connectors contacting the skin after sending the second electrical current.

6. The strain and stimulus sensor system of claim 2, wherein:
each electrical connector of the plurality of electrical connectors includes:
a first connector portion securing the plurality of sensors to the first material, at least some of the first connector portion being positioned on the sensor region to contact the skin of the user; and
a second connector portion secured to at least one wire of the one or more wires and secured or securable to the first connector portion effective to form an electrical connection between the first connector portion and the second connector portion; and
the additional electrical connector includes an additional first connector portion positioned on the sensor region to contact the bony prominence of the user and an additional second connector portion secured to at least one wire of the one or more wires and secured or securable to the additional first connector portion effective to form an electrical connection between the additional first connector portion and the additional second connector portion.

7. The strain and stimulus sensor system of claim 6, further comprising a second material having secured thereto the second connector portion of each connector of the plurality of electrical connectors, the additional second connector portion of the additional connector, and the one or more wires, wherein the second connector portion is detachably securable to the first connector portion and the additional second connector portion is detachably securable to the additional first connector portion such that the second material is detachably securable to the first material.

8. The strain and stimulus sensor system of claim 1, wherein the first material comprises an adhesive and stretchable fabric material.

9. The strain and stimulus sensor system of claim 1, wherein the plurality of sensors are spaced from one another in the sensor region and the plurality of electrical connectors are spaced from one another in the sensor region.

10. A method of collecting strain and stimulus data from a selected region of a body of a user, the method comprising:
securing a first material to the body of a user such that a plurality of electrical connectors secured to the first material contact skin in the selected region of the body, the first material including a sensor region having a plurality of sensors that are stretchable and secured thereto with the plurality of electrical connectors;
sending an electrical current from a circuit board, through one or more wires operably connected to the circuit board and the plurality of electrical connectors, and through the plurality of sensors;
measuring an electrical resistance of the plurality of sensors responsive to the electrical current, the plurality of sensors exhibiting a change in electrical resistance as a function of mechanical deformation; and
measuring an electrical activity at the skin from one or more electrical connectors of the plurality of electrical connectors contacting the skin, the plurality of electrical connectors capturing stimulus data responsive to the electrical current.

11. The method of claim 10, further comprising:
positioning an additional electrical connector to contact a bony prominence of the user, the additional electrical connector being secured to the first material and at least one wire of the one or more wires; and
measuring an electrical activity at the bony prominence from the additional electrical connector contacting the bony prominence.

12. The method of claim 11, further comprising transmitting, from the circuit board, the stimulus data at the bony prominence, the electrical resistance of the plurality of sensors responsive to the electrical current, and the electrical activity at the skin from one or more of the plurality of electrical connectors contacting the skin to an electronic device.

13. The method of claim 12, further comprising determining, with the electronic device, a strain and a stimulus on the user based on the electrical resistance, the electrical activity at the skin from the one or more of the plurality of electrical connectors, and the electrical activity at the skin from the additional electrical connector.

14. The method of claim 13, wherein sending an electrical current from a circuit board, through one or more wires operably connected to the circuit board and the plurality of electrical connectors, and through the plurality of sensors includes:
sending a first electrical current at a first selected frequency through the plurality of sensors; and
sending a second electrical current at a second selected frequency different from the first selected frequency through the plurality of sensors.

15. The method of claim 14, wherein:
measuring an electrical resistance of the plurality of sensors responsive to the electrical current includes:
measuring a first electrical resistance of the plurality of sensors responsive to the first electrical current; and
measuring a second electrical resistance of the plurality of sensors responsive to the second electrical current; and
measuring an electrical activity at the skin from one or more electrical connectors of the plurality of electrical connectors contacting the skin includes:
measuring a first electrical activity at the skin from one or more of the plurality of electrical connectors contacting the skin between sending the first electrical current and the second electrical current; and
measuring a second electrical activity at the skin from one or more of the plurality of electrical connectors contacting the skin after sending the second electrical current.

16. The method of claim 11, further comprising:
for each electrical connector of the plurality of electrical connectors, detachably securing a first connector portion to a second connector portion to form an electrical connection between the first connector portion and the second connector portion, wherein the first connector portion secures a sensor of the plurality of sensors to the first material with at least some of the first connector portion contacting the skin of the user and the second connector portion is secured to at least one wire of the one or more wires; and
detachably securing an additional first connector portion of the additional electrical connector to an additional second connector portion of the additional electrical connector to form an electrical connection between the additional first connector portion and the additional second connector portion, the first connector portion secured to the first material and contacting the bony prominence of the user, and the additional second connector portion secured to at least one wire of the one or more wires.

17. The method of claim 16, wherein the second connector portion of the plurality of electrical connectors and the additional second connector portion of the additional electrical connector are secured to a second material such that detachably securing the first connector portion to the second connector portion and detachably securing the additional first connector portion of the additional electrical connector to the additional second connector portion detachably secures the second material to the first material, wherein the one or more wires are secured to the second material.

18. The method of claim 10, wherein the first material comprises an adhesive and stretchable fabric material.

19. The method of claim 10, further comprising motion capturing the selected region of the body of the user and identifying a plurality of locations for the plurality of sensors based on the motion capturing.

20. The method of claim 10, wherein the plurality of sensors are spaced from one another in the sensor region and the plurality of electrical connectors are spaced from one another in the sensor region.
